(19) United States
(12) Patent Application Publication
(10) Pub. No.: US 2025/0000346 A1
(43) Pub. Date: Jan. 2, 2025

(54) FLEXIBLE SCOPE DEVICE

(71) Applicants: Hiang Khoon TAN, Singapore (SG); VIVO SURGICAL PRIVATE LIMITED, Singapore (SG); DUKE UNIVERSITY, Durham, NC (US)

(72) Inventors: Hiang Khoon TAN, Singapore (SG); Rongsheng Kevin KOH, Singapore (SG); Siang-Long Richard LIEU, Singapore (SG); Janani VENKATESAN, Singapore (SG); Walter Tsong LEE, Hillsborough, NC (US)

(21) Appl. No.: 18/705,155

(22) PCT Filed: Oct. 28, 2022
(86) PCT No.: PCT/SG2022/050784

§ 371 Date: Apr. 26, 2024

(30) Foreign Application Priority Data
Oct. 28, 2021 (SG) 10202111999X

(51) Int. Cl. A61B 1/005; A61B 1/00; A61B 1/008; A61B 1/05

(52) U.S. Cl. CPC A61B 1/0052; A61B 1/00048; A61B 1/00057; A61B 1/0057; A61B 1/008; A61B 1/05

(57) ABSTRACT

FLEXIBLE SCOPE DEVICE There is provided a portable medical endoscope comprising: a flexible portion having a proximal end and a distal end, wherein the flexible portion comprises a bending section and a tip at the distal end, and wherein the tip comprises an image-capturing device; a controller portion disposed at the proximal end of the flexible portion, wherein the controller portion comprises: (i) an angulation mechanism coupled to the bending section and the tip, wherein the angulation mechanism controls movement of the tip; and (ii) a handle in a pistol grip configuration; and a detachable monitor configured to releasably engage the controller portion; wherein the flexible portion and the controller portion are integrally formed, waterproof, and capable of withstanding high level disinfection conditions.

This application claims the benefit of priority of Singapore application No. 10202111999X, filed 28 Oct. 2021, the contents of it being hereby incorporated by reference in its entirety for all purposes.

The invention relates to a flexible scope. In particular, the invention relates to a low-cost, portable and reusable flexible endoscope system.

In developing countries, most patients are not able to undergo complete Head and Neck Cancer (HNC) examinations at district and provincial clinics/hospitals due to the lack of proper equipment at these primary care facilities. Patients are referred to national and central hospitals for basic examinations, often causing overcrowding and overburdening at these tertiary level facilities. At these tertiary care centres, most are found to have issues that could have been treated or addressed at their local facility. Furthermore, treatment for HNC patients that require tertiary level treatment may be delayed due to travel time, costs, and wait times as a result of the overburdening at national and central facilities.

Currently, reusable flexible endoscope systems at tertiary medical centres cost around $50,000 or more, and are large and heavy, limiting their use to a single room. Although such systems are used as a standard of care in clinical examinations for cancers in developed countries, this is not the case in Low-Middle Income Countries (LMICs) due to the flexible endoscope systems' high cost.

A typical alternative solution to the expensive and bulky reusable flexible endoscope systems would be the use of disposable flexible endoscopes connected to a video pad. The disposable flexible endoscopes come at a more affordable initial CAPEX than their reusable counterparts. However, these disposable endoscopes have a disposable single-use portion which would make these systems more expensive and not environmentally sustainable when compared to the reusable flexible endoscopes in the long run, and therefore these disposable endoscopes are not suitable for LMICs.

Usually in LMICs, clinicians use rigid endoscope systems which are slightly more affordable than flexible endoscope systems. However, due to the rigid nature of the scope, these lack the comfort, versatility and ease of use when compared to the flexible ones. Rigid scopes typically consist of a metal shaft with an angled lens at the end of the scope that facilitates views of the oropharynx and larynx during examination. The rigid scope is inserted through the mouth while the patient protrudes their tongue that is held by the health care provider. Examination via a rigid endoscopic has significant drawbacks for both the patient and the examining physician, including: 1) stimulation of the gag reflex in the majority of patients, 2) a limited view of the upper aerodigestive tract due to the fixed angle lens of the scope, 3) restricted examination of laryngeal function due to the retraction of the tongue by the health care provider, and 4) extreme difficulty examining the nasal cavity and nasopharynx for tumours.

Thus, what is needed is a low-cost, portable and reusable flexible endoscope system. Furthermore, other desirable features and characteristics will become apparent from the subsequent detailed description and the appended claims, taken in conjunction with the accompanying drawings and this background of the disclosure.

In one aspect, the present invention provides a portable medical endoscope comprising: a flexible portion having a proximal end and a distal end, wherein the flexible portion comprises a bending section and a tip at the distal end, and wherein the tip comprises an image-capturing device; a controller portion disposed at the proximal end of the flexible portion, wherein the controller portion comprises: (i) an angulation mechanism coupled to the bending section and the tip, wherein the angulation mechanism controls movement of the tip; and (ii) a handle in a pistol grip configuration; and a detachable monitor configured to releasably engage the controller portion; wherein the flexible portion and the controller portion are integrally formed to form a single article such that said single article is waterproof, and capable of withstanding high level disinfection conditions.

In one embodiment, the bending section comprises a plurality of vertebrae.

In one embodiment, the plurality of vertebrae are coupled together by hinges.

In one embodiment, the tip is made of stainless steel.

In one embodiment, the stainless steel is SUS304.

In one embodiment, the image-capturing device comprises a complementary metal-oxide-semiconductor (CMOS) sensor.

In one embodiment, the bending section is made of a precipitation hardening stainless steel.

In one embodiment, the precipitation hardening stainless steel is SUS631.

In one embodiment, the bending section is covered by a bending rubber.

In one embodiment, the bending rubber is made of a high-performance fluoroelastomer.

In one embodiment, the high-performance fluoroelastomer is Viton.

In one embodiment, the controller portion further comprises a pressure valve for measuring an internal pressure of the controller portion.

In one embodiment, the detachable monitor is an LCD monitor.

In one embodiment, the detachable monitor comprises a rechargeable battery.

In one embodiment, the angulation mechanism comprises: a lever configured to control the angulation of the tip; a crank shaft coupled to the lever and to a pair of angulation wires, wherein the pair of angulation wires extend from the controller portion to the tip; wherein a displacement of the lever operates the crank shaft to cause movement of the pair of angulation wires, thereby angulating the tip.

In one embodiment, the angulation mechanism allows the tip to be angulated up to 150 degrees in two directions on a single plane, with a total angulation of 300 degrees.

In one embodiment, the portable medical endoscope as described herein further comprises one or more buttons disposed on the controller portion for activating a function of the image-capturing device.

Advantageously, in addition to being made of medical grade material and catered for ENT usage in terms of size and dimensions, this invention is portable (does not require an endoscope stack), compact (includes a detachable LCD monitor), less expensive (requires less components and less sophisticated equipment that is typically required in clinical settings), durable (being able to withstand high number of bending cycles due to the angulation mechanism described below and use of material selected for this device), chemically resistant (e.g. ability to withstand high level disinfection solutions), and easy to use (due in part to the pistol grip handle configuration).

The invention will be better understood with reference to the detailed description when considered in conjunction with the non-limiting examples and the accompanying drawings, in which:

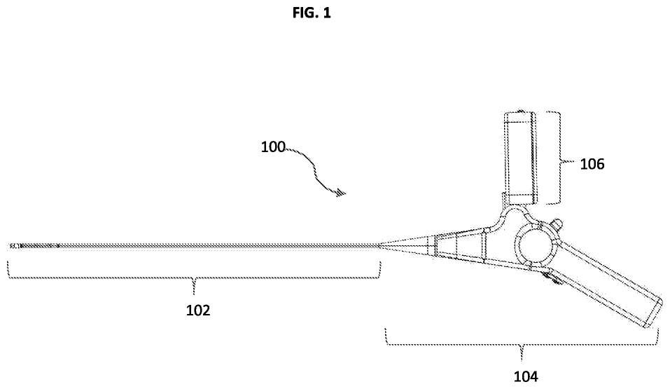

FIG. 1 shows a side planar view of the portable medical endoscope in accordance with present embodiments.

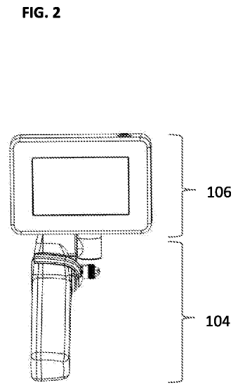

FIG. 2 shows a front planar view of the portable medical endoscope of FIG. 1.

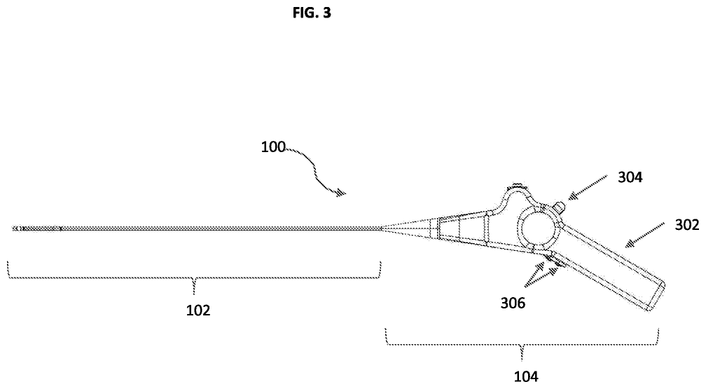

FIG. 3 shows a side planar view of a flexible portion and a controller portion of the portable medical endoscope of FIG. 1.

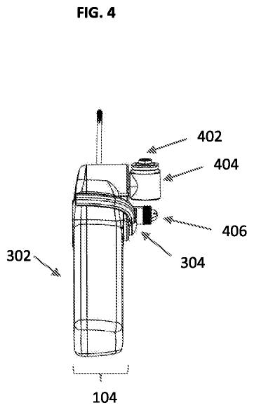

FIG. 4 shows a front planar view of the controller portion of the portable medical endoscope of FIG. 3.

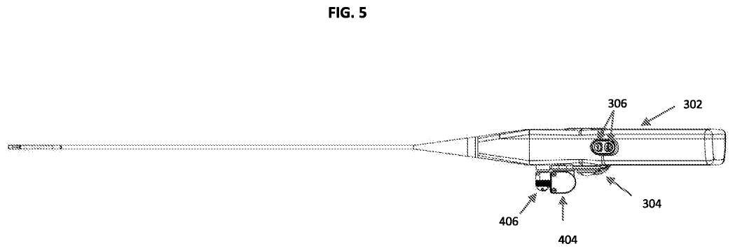

FIG. 5 shows a bottom planar view of the controller portion and the flexible portion of the portable medical endoscope of FIG. 3.

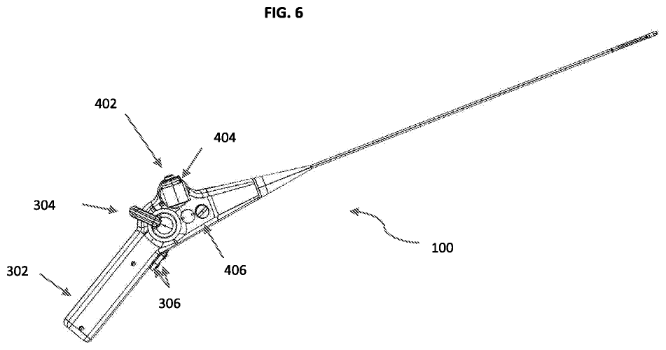

FIG. 6 shows a side planar view of the controller portion and the flexible portion of the portable medical endoscope of FIG. 3.

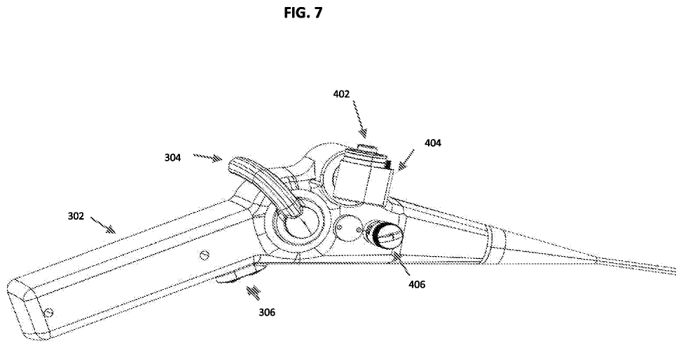

FIG. 7 shows a top side perspective view of the Controller portion of the portable medical endoscope of FIG. 3.

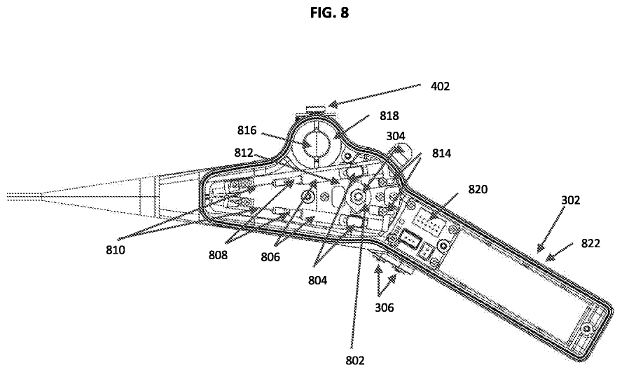

FIG. 8 shows a side sectional view of the controller portion of the portable medical endoscope of FIG. 3.

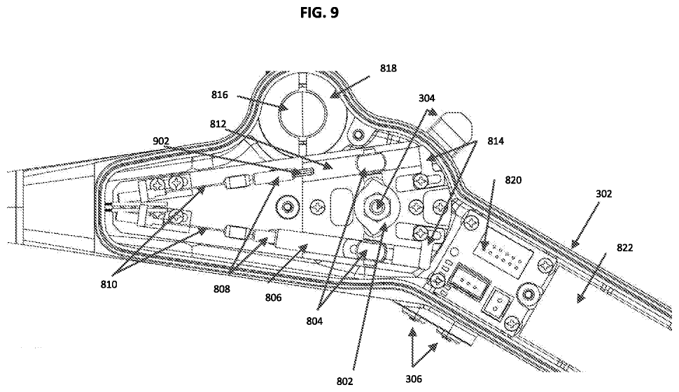

FIG. 9 shows a side sectional view of the angulation mechanism in the controller handle of the portable medical endoscope of FIG. 3.

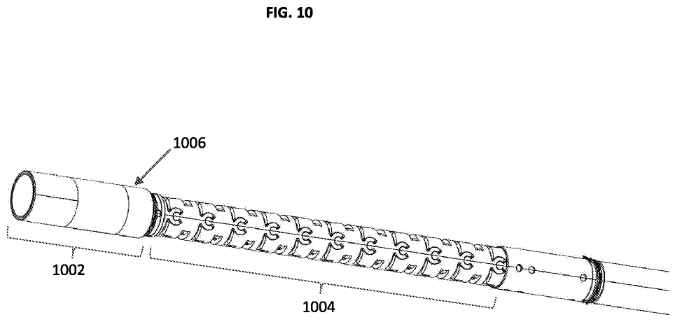

FIG. 10 shows a rear side perspective view of a distal end of the flexible portion of the portable medical endoscope in accordance with the present embodiments, with a distal tip bushing and a bending rubber not shown.

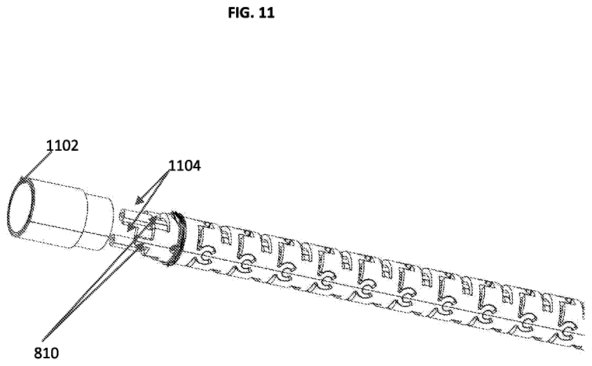

FIG. 11 shows an exploded rear side perspective view of the distal end of the portable medical endoscope in accordance with the present embodiments, with the distal tip bushing and bending rubber not shown.

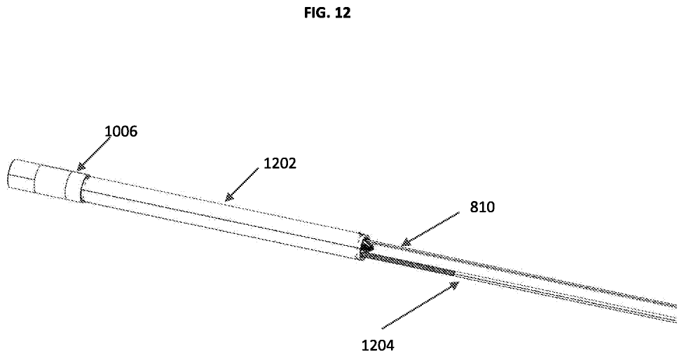

FIG. 12 shows a side view of the flexible portion of the portable medical endoscope in accordance with the present embodiments with a sheath (i.e. outer cover) of the flexible portion not shown.

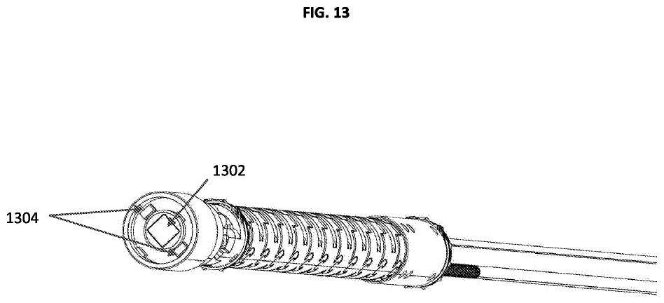

FIG. 13 shows a rear side perspective view of the distal end of the flexible portion of the portable medical endoscope in accordance with the present embodiments, with the distal tip bushing, bending rubber and sheath not shown.

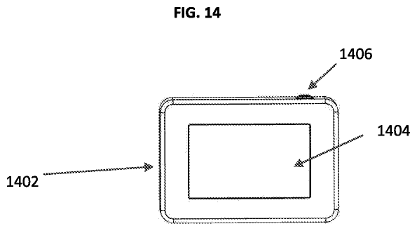

FIG. 14 shows a front planar view of a detachable monitor of the portable medical endoscope in accordance with the present embodiments.

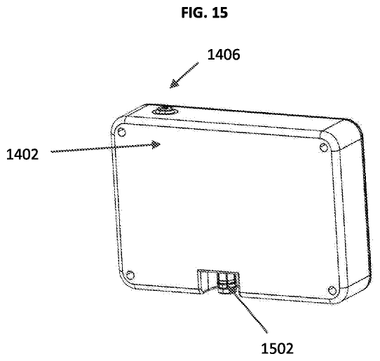

FIG. 15 shows a rear side perspective view of the detachable monitor in accordance with the present embodiments.

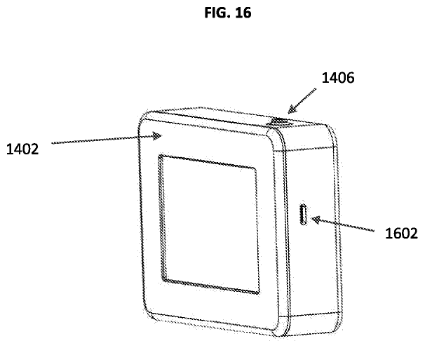

FIG. 16 shows a front side perspective view of the detachable monitor in accordance with the present embodiments.

The following detailed description is merely exemplary in nature and is not intended to limit the invention or the application and uses of the invention. Furthermore, there is no intention to be bound by any theory presented in the preceding background of the invention or the following detailed description. It is the intent of the present embodiment to present a portable medical endoscope that is low-cost and reusable.

The present disclosure provides a portable medical endoscope comprising: a flexible portion having a proximal end and a distal end, wherein the flexible portion comprises a bending section and a tip at the distal end, and wherein the tip comprises an image-capturing device; a controller portion disposed at the proximal end of the flexible portion, wherein the controller portion comprises: (i) an angulation mechanism coupled to the bending section and the tip, wherein the angulation mechanism controls movement of the tip; and (ii) a handle in a pistol grip configuration; and a detachable monitor configured to releasably engage the controller portion, wherein the flexible portion and the controller portion are integrally formed, waterproof, and capable of withstanding high level disinfection conditions.

The device or endoscope of the present disclosure may be used in a variety of endoscopic procedures, including but not limited to nasopharyngeal endoscopy, bronchoscopy, esophagoscopy, gastroscopy or laryngoscopy. Alternatively, it may be used to observe the interior of any bodily orifice. Apart from cancer-related applications, applicable procedures include but are not limited to Pre- and Post-operative Endoscopic Airway Examination (PEAE), Fiberoptic Endoscopic Evaluation of Swallowing (FEES) for speech pathology, Drug Induced Sleep Endoscopy (DISE) for sleep apnoea, checking for Velopharyngeal Dysfunction (VPD) and postoperative examination after Endoscopic Sinus Surgery (ESS).

FIG. 1 shows a side view of the portable medical endoscope 100 (also known as “endoscope” or “device”) in accordance with present embodiments. The flexible portion 102 is also known as a “flexible scope” or “flexible scope portion”. The controller portion 104 is also known as “controller handle”. In one embodiment, the detachable monitor 106 is an LCD monitor

FIG. 2 shows a front view of the portable medical endoscope 100 with the detachable monitor 106 attached.

An embodiment of the portable medical endoscope 100 with the detachable monitor 106 absent is shown in FIG. 3 (left side view), FIG. 4 (front view), FIG. 5 (bottom view) and FIG. 6 (right side view).

Most reusable endoscopes in the prior art have to be connected to an endoscopy stack to be operational, which limits the portability of such prior art endoscopes. Advantageously, the medical endoscope of the present disclosure will be used as a standalone device without the need to be connected to an endoscopy stack; it has a detachable monitor 106 that can be connected to or detached from the controller portion 104, allowing the medical endoscope to be portable. Users can directly review photos and videos on the detachable monitor without having to extract the data to a computer.

### Flexible Portion

The flexible portion 102 is also known as “flexible scope”. As used herein, the term “scope” may be used interchangeably with “probe”, “tube” or “insertion tube”. Such scopes are useful in viewing the interior of an orifice. In one embodiment, the flexible portion 102 is engineered to have a small diameter of below 4 mm, allowing for easy and comfortable insertion into the patient. The diameter of the flexible portion may range from 1 mm to 15 mm. In particular, in various embodiments, the tip of the flexible portion may have an outer diameter of about 3.5 mm. In various embodiments, the insertion tube of the flexible portion 102 may be about 350 mm long.

The flexible portion 102 is adapted to engage with a controller portion 104 in a watertight manner or is integrally formed (i.e. formed in a single article) with the controller portion 104. In a preferred embodiment, the flexible portion 102 is integrally formed with the controller portion 104. Advantageously, upon detaching the detachable monitor 106, the flexible portion 102 and controller portion 104 can be fully submerged in fluid for high level disinfection, allowing the portable medical endoscope 100 to be reusable.

As shown in FIG. 10, the flexible portion 102 comprises a tip 1002 (also known as “scope tip” or “flexible tip”) connected to a bending section 1004 at the distal end. The tip 1002 is covered by a distal tip bushing 1006. The tip 1002 is flexible, may have an outer diameter of about 3.5 mm, and may be angulated up to 150 degrees in 2 directions on a single plane (300 degrees in total), allowing the end user to comprehensively view the body cavity area of interest in a retroflex fashion. This angulation is enabled by the angulation mechanism in the controller portion 104 that has 2 angulation wires 810 connected to 2 angulation stoppers 1104 which are secured to the distal tip bushing 1006 of the flexible tip 1002, as shown in FIG. 11. As shown in FIG. 12, 2 angulation wires 810 are housed inside 2 wire coils 1204 that are used to guide the wires from the controller portion 104 to the flexible tip 1002. Advantageously, the angulation mechanism allows the direction of the tip 1002 to be controlled by the user.

In the present embodiments, the bending section 1004 at the distal end of the flexible portion 104 is engineered in such a way to allow for up to 100 times bending per day for 6 consecutive months totalling more than 18,000 times of bending. The bending of the bending section 1004 allows for the angulation of the tip 1002. The bending section 1004 is also known as the “vertebrae” of the endoscope. Several metal bands are hinged together in a manner that alternates vertebra-air space-vertebra. This configuration enables the vertebrae to move vertically and horizontally. The design of the bending section 1004 allows for low friction when moving in the intended plane but provides high resistance when forces are applied in torsional motion, thus increasing the lifespan of the bending section 1004 as compared to traditional rivet style bending sections. Advantageously, the design of bending section 1004 allows for increased bending cycles, durability and low-cost of manufacturing.

As shown in FIG. 12, the bending section 1004 may be covered by a bending rubber 1202 which is on the exterior of the flexible portion. In addition to the design of the bending section 1004, the materials used to manufacture the bending section 1004 and the bending rubber 1202 also contribute to the high durability of the bending section 1004. In one embodiment, the bending section may be composed of SUS631, a precipitation hardening stainless steel. This type of steel is known to provide significantly higher tensile strength and yield strength as opposed to other types such as the austenitic stainless steel. In one embodiment, the bending rubber 1202 is composed of Viton, a high-performance fluoroelastomer polymer with high tensile strength and tear resistance.

Advantageously, the use of high-performance fluoroelastomer to form the bending rubber 1202 allows for increased durability (high number of bending cycles allowed), chemical resistance to high level disinfection solutions, and increased flexibility of the bending rubber 1202. A high-performance fluoroelastomer is a material that has high resistance to a wide range of chemicals which enables the material to withstand a larger number of disinfection cycles during the reprocessing stage. This contributes to the durability of the bending rubber 1202 and of the overall portable medical endoscope 100.

By “fluoroelastomer”, it is meant to include any highly fluorinated polymer that is suitable for continuous use at elevated temperatures. Fluoroelastomers have excellent heat and flame resistance, and outstanding resistance to aging, ozone, oxidizers, oils and many chemicals. They also have low gas permeability and low compression set.

The tip may be made of stainless steel such as SUS304. By “SUS304”, it is meant to include any “Steel Use Stainless” material containing chromium (between about 18% to 20%) and nickel (between about 8% to 10.5%) as the main non-iron constituents. It can maintain good strength and heat resistance in high temperature and low temperature environments, and also has good corrosion resistance, weldability, cold workability and mechanical properties in the mild atmosphere.

The sheath of the flexible scope portion 102 is made of biocompatible material to enable the device to be used in humans and is designed to enable insertion into the patients' cavity without causing any injury nor discomfort. The biocompatible material does not produce a toxic or immunological response in humans. Examples of biocompatible materials that can be used to make the sheath of the scope portion 102 include thermoplastic polyester elastomer (TPC), thermoplastic polyurethane elastomer (TPU) and polyvinyl chloride (PVC). The sheath of the scope may also be composed of a mixture of TPC and TPU, or a mixture of TPU and TPC containing soft PVC.

The tip 1002 comprises an image-capturing device which may include an illumination source (such as one or more LED modules 1304 shown in FIG. 13), an image sensor (such as a CMOS sensor 1302 shown in FIG. 13) and an objective lens 1102 to allow for capturing of images or videos of the illuminated interior of the orifice or cavity under examination. For example, a high-resolution CMOS sensor 1302 may be housed behind a lens 1102 at the tip 1002, which would ensure a clear field of view and enable easy identification of any abnormal tissue features during screening. In various embodiments, surrounding the CMOS are high powered, high Colour Rendering Index (CRI) and high R9 value LED modules 1304 that enable the tip of the scope to emit sufficient light output at the tip 1002 of the flexible portion 102 for the end user to view the patient's tissue and to ensure that the tissue is viewed with a good quality of light. This allows the user to make better tissue identification and to make faster and more accurate diagnosis. A suitable and adequate heat sink or heat transfer system may be used to transfer thermal energy away from the tip 1002, for example using heat pips for transferring the thermal energy to the controller handle. Advantageously, the device of this invention does not require a separate light guide or light source from an endoscope stack through the flexible portion 102. Light guides have been known to be more fragile and will deteriorate during re-processing and/or sterilization. On the other hand, the LED modules 1304 at the tip 1002 have been selected to have the following features high powered, high Colour Rendering Index (CRI) and high R9 value to better enhance the tissue that the camera is focusing on.

The flexible portion 102 is designed, and materials are chosen in such a way to allow it to be waterproof and to be able to withstand long-term harsh chemical high-level disinfection durations, to prevent cross contamination between patients after each use. Chemicals that may be used for high-level disinfection include but are not limited to glutaraldehyde, hydrogen peroxide, peracetic acid and ortho-phthalaldehyde. Examples of trade names of commonly used high level disinfectants that may be used with the device of the present invention include Cidex PLUS (glutaraldehyde), Cidex OPA (phthalaldehyde), Aldahol (glutaraldehyde), Rapicide (peracetic acid), Resert XL (hydrogen peroxide) and Acecide (peracetic acid and hydrogen peroxide).

The flexible portion 102 is connected to the controller portion 104 at the proximal end.

### Controller Portion

The controller portion 104 is also known as “controller handle”. As shown in FIGS. 7-9, the controller portion 104 includes a pistol grip 302 (also known as “pistol grip configuration”), an angulation mechanism, a pressure valve 406, a tilt mechanism 404 for tilting the detachable monitor 106, a waterproof connector 402 for connecting to the detachable monitor 106, 2 buttons 306 for performing photo-taking and/or video-taking functions, and electronic components such as a camera driver module 820 and back-up battery 822.

The controller portion 104 enables the end-user to control the tip 1002 of the endoscope to achieve angulation up to 150 degrees in 2 directions on a single plane (300 degrees in total). This allows the end-user to better steer the endoscope tip 1002 in the desired direction during scope introduction and also allows for comprehensive viewing in the body cavity of interest.

The controller portion 104 is also designed, and materials are chosen in such a way to allow it to be waterproof and to be able to withstand long-term harsh chemical high-level disinfection durations, so as to prevent cross contamination between patients after each use. By “high level disinfection”, it is meant to refer to any treatment of medical devices and instruments that inhibits most viable microorganisms. Chemicals that may be used for high-level disinfection include but are not limited to glutaraldehyde, hydrogen peroxide, peracetic acid and ortho-phthalaldehyde. Examples of trade names of commonly used high level disinfectants that may be used with the device of the present invention include Cidex PLUS (glutaraldehyde), Cidex OPA (phthalaldehyde), Aldahol (glutaraldehyde), Rapicide (peracetic acid), Resert XL (hydrogen peroxide) and Acecide (peracetic acid and hydrogen peroxide).

The device itself is designed and built to be robust to withstand in the field (outside the clinic setting) type of work.

In a vast majority of endoscopes, the handle on the controller portion 104 is in the form of a conventional power grip. In the present invention, the handle comprises a pistol grip configuration 302. The ergonomic design of the pistol grip 302 allows for improved control and ease of use. In addition, the pistol grip 302 is more comfortable and intuitive for new users and may therefore accelerate the adoption of the device. The anti-slip texture at the bottom and top portion of the handle improves the grip of the endoscope 100 when the end user holds onto it. The controller portion 104 has also been designed such that the centre of gravity of the controller portion 104 sits nicely at the palm of the end users for improved comfort when using the endoscope and when the detachable monitor 106 is plugged into the controller portion 104. The controller portion 104 has also been designed with a handle grip angle of between 45-90 deg for improved comfort and ease of use. The handle grip angle is the degree the pistol grip configuration 302 is bent in relation to the horizontal axis. In other words, it is the angle formed between the horizontal plane and the central axis of the pistol grip configuration 302. The combination of these features minimizes ergonomic stress experienced by users, especially during device use across prolonged periods.

The controller portion 104 comprises a lever 304 on the top side of the pistol grip 302 that is intuitive to use and easy to use for controlling the scope tip 1002 angulation via a push and pull mechanism with the end user's finger. As shown in FIGS. 8 and 9, the lever 304 is connected to a crank shaft 802 which is in turn attached to 2 connector rods 804. Each connector rod 804 is secured to a sliding rod 806. A wire stopper 808 and a wire soldering rod 902 are incorporated inside each sliding rod. An angulation wire 810 is affixed to each wire stopper 808 and wire soldering rod 902. Consequently, pulling or pushing the lever 304 would cause movement of the angulation wire 810, which in turn would enable the flexible tip 1002 to bend.

For example, the pulling of the lever 304 activates the crank shaft 802, which in turn causes movement of the connector rod 804, sliding rod 806, wire stopper 808 and angulation wire 810 (in the order mentioned). As the angulation wires 810 are connected to the distal tip bushing 1006, the pulling or pushing of the lever 304 results in the bending of the bending section 1004, thereby angulating the tip 1002. The extent of lever movement is proportional to the angle of deflection at the tip 1002, and a larger movement of the lever 304 corresponds to a larger angulation of the tip 1002. The movement of each sliding rod 806 will be controlled by an angulation base 812 and angle stopper 814. The angulation base 812 and angle stopper 814 are in place so that the sliding rod 806 has a restricted space to move in-between and therefore the lever 304 will be able to push and pull the angulation wires 810 up and down. In one embodiment, the angulation mechanism allows the tip 1002 to be angulated up to 150 degrees in two directions (300 degrees in total).

Advantageously, the angulation mechanism comprising the crank shaft 802 as described herein allows the movement of the angulation wires 810 to be more stable during angulation of the tip 1002 and is therefore superior over existing angulation mechanisms that comprise conventional pulley systems.

In one embodiment, the device also incorporates buttons 306 disposed on the controller portion 104 for activating a function of the image-capturing device. By “activating a function”, it is meant to include activating photo-taking and/or video-taking functions. The buttons 306 can also be used to scroll between images or videos when accessed in playback mode. These buttons 206 may be used for easy photo and video taking activation. All other functions during playback mode may be performed via the touchscreen feature of the monitor 106. In addition, while possible to do so, playback may be carried out directly from the LCD monitor without having to extract the data to a computer. For example, the buttons 306 may be located at the bottom side of the pistol grip 302 for ease of use when the end user wants to perform photo-taking and/or video-taking functions. The buttons 306 are embossed with 3-dimensional ‘X’ and ‘O’ symbols to allow the user to differentiate between the photo and video taking buttons in a tactile manner. It would be generally understood by a person skilled in the art that the buttons 306 could be located elsewhere on the controller portion 104.

The controller portion 104 is equipped with a tilt mechanism 404 which allows the detachable monitor 106, when plugged into the controller portion 104, to be able to tilt from front to back to optimize the viewing angle. This tilt mechanism 404 allows the end user to easily change the viewing angle of the detachable monitor 106 to the most comfortable and ideal angle. The tilt mechanism 404 is controlled by a connector hinge 816 which is attached to a hinge retainer 818. The hinge retainer 818 determines the detachable monitor's minimum and maximum tilt angulation vis-à-vis the controller portion 104.

The controller handle is equipped with a pressure valve 406 which allows the end user to check the internal pressure inside the controller portion 104 before usage. To check the internal pressure, the user connects a pressure pump to the controller portion 104, pumps air into the controller portion 104 until a certain internal pressure is reached, and leaves it for 2 to 3 minutes. In one embodiment, the internal pressure after pumping ranges from 140 mmHg to 200 mmHg. The subsequent stabilization of the pressure without any rapid decline in pressure indicates that the controller portion 104 has no leak. A drop in pressure is indicative of one or more leaks in the controller portion 104.

The controller portion 104 houses some of the electronic components such as the camera driver module 820.

The controller handle is also equipped with a waterproof connector 402, which may also be known as a “female connector”, that allows connectivity between the detachable monitor 106 and the controller portion 104. This connector feature has been designed to be waterproof. In particular, the female connector 402 is sealed to the controller handle, and the capping of the female connector 402 renders it waterproof. Additionally, components such as an O-ring are used to ensure a tight fit and waterproofing capabilities between the female connector 402 and the controller portion 104. This enables the controller portion 104 to be soaked by the end user in harsh chemicals for high level disinfection purposes.

### Detachable Monitor

The detachable monitor 106 may be an LCD monitor. In the embodiment shown in FIG. 14, the detachable monitor 106 comprises an LCD monitor electronics housing 1402, an “on/off” button 1406, and a touchscreen 1404. In the rear side perspective view of the detachable monitor 106 shown in FIG. 15, a waterproof connector 1502.

The detachable monitor 106 is built to incorporate touchscreen function which provides for ease of use to the end user as well as familiarity with the system. The end user is able to tap the screen with a light touch of their fingers, or while wearing gloves, to enable various functionalities such as choosing to start a new scoping session, for reviewing past recorded media or choosing the device code name, which allows the user to differentiate this particular device from another. The detachable monitor 106 is also equipped with a connector 1502, also known as a “male connector” (shown in FIG. 15), that is adapted to engage with the female connector 402 on the controller portion 104 and allows connectivity between the detachable monitor 106 and the controller portion 104.

Advantageously, the detachable monitor 106 can be connected to the controller portion 104 directly without requiring a cable.

The signal from the camera module driver 820 is transmitted from the camera module driver 820 of the controller portion 104 to a mainboard housed in the detachable monitor 106 and subsequently to the screen 1404 of the monitor, thereby displaying an image on the screen. Despite featuring a touchscreen 1404, the detachable monitor 106 is also built to withstand wipe-downs with alcohol solutions so as to prevent cross contamination between patients after each use.

Apart from immediate viewing of live camera images on a planned compact 4″ (or any suitably sized monitor such as 3.5″) built-in high-resolution LCD monitor (‘live mode’), the device is designed to incorporate media (photo and video) recording and playback functions (‘review mode’). The monitor resolution may be at least 400×400 for clear visualization. This allows clinicians to re-evaluate or review the captured images/videos at a later time or to share any key findings after the procedure. Users can also toggle between ‘live’ and ‘review’ modes easily. The media recorded by the device can be transferred via USB, such as via USB port 1602 shown in FIG. 16, to a computer for later viewing as well. USB port 1602 may be a USB-C port for charging and for data transfer/export.

During scoping sessions, white balance is critical for the end user to effectively view the tissues in patients. In the present invention, the white balance feature is automated.

Overall, the device is constructed to contain modular parts that can be easily removed and assembled up to 5,000 times thanks to its deliberate plug and play design mechanism between the detachable monitor 106 and the controller portion 104. The term “plug and play” refers to the feature of the device being ready to use immediately when first connected, without requiring reconfiguration or adjustment by the user. Such modularity also renders flexibility to the user in switching between different scopes (for example, flexible scopes and portions, and handles) for various use cases and different models of screens when future generations are made available. The detachable monitor 106 may be used with different versions of the controller portion 104 which may be attached or connected to flexible portions 102 having different lengths, tip 1002 diameters etc. so as to provide for the possibility of catering to different surgical specialties other than the head and neck cancers that may be used for this invention. Also, advantageously, this invention does not have any ancillary equipment (such as suction pump and biopsy forceps). In other words, unlike conventional endoscopes, the device of this invention does not have a “working channel” or lumen for containing ancillary equipment which makes it easy to use and maintain, and portable. Here, there is no need for reprocessing machines to clean and disinfect the insides of the lumen of conventional endoscopes.

The detachable monitor 106 includes rechargeable batteries, allowing for the endoscope 100 to be used continuously for up to 4 hours, which is longer than the typical 30 minutes of usage duration in similar handheld endoscope devices in the prior art. Beneficially, the lengthened duration of use per charge also increases the suitability of the present endoscope 100 for fieldwork. The endoscope 100 can be charged via a USB port 1602 on the detachable monitor 106. The battery life of the device may be displayed on the monitor 106 for efficient planning of device usage.

### CONCLUSION

Thus, it can be seen that the present disclosure provides a flexible medical endoscope that is portable, low-cost and reusable.

Advantageously, the portable, low-cost and reusable endoscope 100 of the present disclosure facilitates patient examination by doctors in primary care settings at local district and provincial hospitals. As such, it allows for early diagnosis of cancers, even in less economically developed countries. Furthermore, patient care burden at national specialty hospitals can be greatly reduced, with attention refocused to conditions that truly require tertiary care (i.e. advanced cancer).

Furthermore, the pistol grip 302 of the endoscope of the present disclosure is more ergonomic for the user as compared to the conventional power grip that is present in the majority of endoscopes currently on the market. Furthermore, the pistol grip 302 allows for improved control and ease of use of the device. In particular, the pistol grip 302 is more comfortable and intuitive for new users. Pistol grips 302 are suitable when the force is applied in the same direction as the extended forearm and wrist, such as in the case of the endoscope of the present disclosure. The ergonomic advantages of the pistol grip 302 over the power grip may be attributed to the lower ergonomic stress required for supination of the pistol grip, handle grip angle of between 45-90 deg, less power required to hold the endoscope, and reduced force required to manipulate the control lever. Compared to the power grip, the use of a pistol grip 302 allows the flexible medical endoscope 100 to be less bent and more linear during examination. This potentially reduces friction between the endoscope 100 and the nasopharyngeal mucosa, allowing for more efficient manipulation of the endoscope 100.

Another important advantage is that the bending section 1004 at the distal end of the flexible portion 102 is engineered to withstand frequent bending without damage to the bending section 1004. This advantage of high durability of the bending section 1004 is in part conferred by the materials used to manufacture the bending section 1004 and the bending rubber 1202. As such, the medical endoscope 100 of the present disclosure can be reused multiple times before requiring replacement of the bending rubber 1202. The risk of fluid invasion due to holes and tears in the bending section 1004 is also minimised.

Another advantage of the endoscope of the present disclosure is that the materials used and the design of the external portion of the flexible portion 102 and controller portion 104 are waterproof and are connected in a watertight manner so as to allow the device to undergo high level disinfection soaking during the reprocessing procedure. As such, detaching the monitor 106 from the controller portion 104 allows the entire flexible portion 102 and controller portion 104 to be submerged in fluid for high level disinfection. In contrast to single-use endoscopes, which require some parts to be disposable due to the inability to of these parts to withstand high level disinfection processes, the endoscope 100 of the present disclosure is able to maintain waterproof integrity and can be fully reprocessed after each use.

While embodiments of the invention have been particularly shown and described with reference to specific embodiments, it should be understood by those skilled in the art that various changes in form and detail may be made therein without departing from the scope of the invention as defined by the appended claims. The scope of the invention is thus indicated by the appended claims and all changes which come within the meaning and range of equivalency of the claims are therefore intended to be embraced.

1. A portable medical endoscope comprising:
a flexible portion having a proximal end and a distal end, wherein the flexible portion comprises a bending section and a tip at the distal end, and wherein the tip comprises an image-capturing device;
a controller portion disposed at the proximal end of the flexible portion, wherein the controller portion comprises:
(i) an angulation mechanism coupled to the bending section and the tip, wherein the angulation mechanism controls movement of the tip; and
(ii) a handle in a pistol grip configuration; and
a detachable monitor configured to releasably engage the controller portion;
wherein the flexible portion and the controller portion are capable of withstanding high level disinfection conditions.

2. The portable medical endoscope of claim 1, wherein the bending section comprises a plurality of vertebrae.

3. The portable medical scope device of claim 2, wherein the plurality of vertebrae are coupled together by hinges.

4. The portable medical endoscope of claim 1, wherein the tip is made of stainless steel.

5. The portable medical endoscope of claim 4, wherein the stainless steel is SUS304.

6. The portable medical endoscope of claim 1, wherein the image-capturing device comprises a complementary metal-oxide-semiconductor (CMOS) sensor.

7. The portable medical endoscope of claim 1, wherein the bending section is made of a precipitation hardening stainless steel.

8. The portable medical endoscope of claim 7, wherein the precipitation hardening stainless steel is SUS631.

9. The portable medical endoscope of claim 1, wherein the bending section is covered by a bending rubber.

10. The portable medical endoscope of claim 9, wherein the bending rubber is made of a high-performance fluoroelastomer.

11. The portable medical endoscope of claim 10, wherein the high-performance fluoroelastomer is Viton.

12. The portable medical endoscope of claim 1, wherein the controller portion further comprises a pressure valve for measuring an internal pressure of the controller portion.

13. The portable medical endoscope of claim 1, wherein the detachable monitor is an LCD monitor.

14. The portable medical endoscope of claim 1, wherein the detachable monitor comprises a rechargeable battery.

15. The portable medical endoscope of claim 1, wherein the angulation mechanism comprises:
a lever configured to control the angulation of the tip;
a crank shaft coupled to the lever and to a pair of angulation wires, wherein the pair of angulation wires extend from the controller portion to the tip;
wherein a displacement of the lever operates the crank shaft to cause movement of the pair of angulation wires, thereby angulating the tip.

16. The portable medical endoscope of claim 1, wherein the angulation mechanism allows the tip to be angulated up to 150 degrees in two directions on a single plane, with a total angulation of 300 degrees.

17. The portable medical endoscope of claim 1, wherein the flexible portion and the controller portion are integrally formed to form a single article capable of withstanding high level disinfection conditions.

18. The portable medical endoscope of of claim 1, further comprising one or more buttons disposed on the controller portion for activating a function of the image-capturing device.
